(19) United States
(12) Patent Application Publication
(10) Pub. No.: US 2013/0030266 A1
(43) Pub. Date: Jan. 31, 2013

(54) PSYCHOLOGICAL STRESS INDEX MEASURING SYSTEM AND ANALYSIS METHOD

(71) Applicants: Jiann-Shing SHIEH, Chung-Li City (TW); Maysam F. Abbod, Brunel University (GB); Wan-Ting Chiang, Chung-Li City (TW); Si-Hui Yang, Chung-Li City (TW)

(72) Inventors: Jiann-Shing SHIEH, Chung-Li City (TW); Maysam F. Abbod, Brunel University (GB); Wan-Ting Chiang, Chung-Li City (TW); Si-Hui Yang, Chung-Li City (TW)

(21) Appl. No.: 13/193,879

(22) Filed: Jul. 29, 2011

(51) Int. Cl. A61B 5/1455

(52) U.S. Cl. 600/323

(57) ABSTRACT

A psychological stress index measuring system and its analysis method comprise a pulse generator, a transmission-based photo sensor, a signal converter & amplifier, a filter and a processing platform. At first, the transmission-based photo sensor fixed on a user depends on a light transmitting mechanism to detect analog SpO₂ (oxyhemoglobin saturation by pulse oximetry) signals. Then, it will be processed and transformed to digital signals and further exported to the processing platform for extraction of PPGA & HBI. Finally, it will pass a median filter, normalization, and development of Psychological Stress Index which is based on the normalized PPGA & HBI as one convenient method to evaluate psychological health.

### BACKGROUND OF THE INVENTION

1) Field of the Invention

The present invention relates to a psychological stress index measuring system and its analysis method, especially to a measuring system based on a noninvasive physiological signal measuring device to acquire oxyhemoglobin saturation by pulse oximetry (SpO2) signals and further analyze the signals thereof for the psychological stress index.

2) Description of the Prior Art

In an environment full of social changes, an individual playing more and more roles has to sustain considerable psychological stress in a personal life generally. It is getting more important to keep an individual's psychological health because these psychological factors of one individual in the face of chronic dysphoria or anxiety will adversely affect healthy or even cause any disease threatening life. Currently, the common methods to evaluate a person's psychological status comprise questionnaire survey and psychological consultation. On the other hand, the physiological signals based on expensive specialized instruments are also available such as electroencephalogram and multi-functional physiological monitor;

As a result, a variety of methods to evaluate psychological status have been applicable in different fields at present. For instance, the article of “Assessment of Surgical Stress during General Anaesthesia” published by Huiku et al. in 2007 mentioned that various physiological signals such as Plethysmograph amplitude (PPGA), Heart Beat Interval (HBI), and Pulse Transit Time (PTT) detected at one patient who sustained one of two different types of simulations, either incision or anesthetic, during surgery were affected and changed due to any surgical stress. In his conclusions, Huiku et al. found a significant correlation between PPGA and HBI in terms of surgical stress by one patient during surgery. Therefore, they took PPGA and HBI to analyze Surgical Stress Index (SSI). Also, Huiku et al. applied for one U.S. patent (U.S. Pat. No. 7,635,337 B2, Determination of Clinical Stress of a Subject in Pulse Oximetry) in which there is one pulse oximeter used to detect any breath-induced plethysmograph signal for further defining stress sustained by one patient during surgery;

Additionally, other patents with respect to evaluation of psychological status are R.O.C. Patent No. M389304, M312974, and 1311067 or U.S. Pat. No. 7,613,486, U.S. Pat. No. 7,282,028, U.S. Pat. No. 7,547,279, and U.S. Pat. No. 7,367,949. In this regard, three R.O.C. patents (M389304; M312974; 1311067) mentioned oxyhemoglobin saturation by pulse oximetry (SPO₂), finger temperature, heartbeat, and skin conductance taken as evaluative indicators without any method to evaluate a person's psychological status distinctly. They all indicated or described some conceptual ideas without any psychological stress level explicitly quantified so that any status of a person's psychological stress is not comprehensively understood owing to their unclear expressions.

On the other hand, it can be seen from four U.S. patents (U.S. Pat. No. 7,613,486; U.S. Pat. No. 7,282,028; U.S. Pat. No. 7,547,279; U.S. Pat. No. 7,367,949) that the emotional status could be analyzed with various physiological variables such as heartbeat, SpO₂, skin temperature, etc. measured according to current technologies. Hence, it can be seen from the said four patents that most methods are intended for analyzing emotional status of patients whose emotional changes are rarely distinctive and significantly different from those of general people or for measuring patients' pains or sedation, so as to be unavailable to the general public. Despite some patents with their objects not clearly specified, these patents previously mentioned do not contribute to practical values obviously because any definite quantitative analysis is not available but some levels from negative emotion to positive emotion are roughly differentiated to obtain a tendency of significant emotional changes.

It is very inconvenient for the elders or patients who depend on medical appliances for a long period to use most existing invasive medical appliances currently in measurement of psychological signals. In this context, a psychological stress index measuring system and its analysis method based on a noninvasive measuring device for measurement of SpO₂ signals by which any psychological stress index is developed and any psychological stress level is quantified could be regarded as the optimal solution.

### SUMMARY OF THE INVENTION

The object of the present invention is to provide a psychological stress index measuring system and its analysis method based on noninvasive measurement of oxyhemoglobin saturation by pulse oximetry (SpO₂) signals to develop an index of evaluating psychological stress index and quantify psychological stress levels.

The other object of the present invention is to provide a psychological stress index measuring system and its analysis method with a noninvasive system to measure any oxyhemoglobin saturation by pulse oximetry (SpO₂) signal for the purpose of convenience, portability, and operability.

In the present invention of a psychological stress index measuring system and its analysis method delivering the said objects, the psychological stress index measuring system comprises a pulse generator, a transmission-based photo sensor, a signal converter & amplifier, a filter and a processing platform. The transmission-based photo sensor fixed on a user's finger features a light transmitting mechanism applied to detect any analog SpO₂ signal which is processed and transformed to a digital signal and further exported into the processing platform for extraction of PPGA & HBI. The analyses such as median filter and normalization, and normalized PPGA & HBI are transferred to a psychological stress index as one ultra-simple method to evaluate or measure psychological health.

### BRIEF DESCRIPTIONS OF THE DRAWINGS

FIG. 1 illustrates the overall structure for the present invention of a psychological stress index measuring system and its analysis method;

FIG. 2 illustrates the analytic flowchart for the present invention of a psychological stress index measuring system and its analysis method;

FIG. 3 illustrates definitions of PPGA & HBI for the present invention of a psychological stress index measuring system and its analysis method;

FIG. 4A illustrates extracted PPGA for the present invention of a psychological stress index measuring system and its analysis method;

FIG. 4B illustrates extracted HBI for the present invention of a psychological stress index measuring system and its analysis method;

FIG. 5A illustrates a flowchart with respect to normalization for the present invention of a psychological stress index measuring system and its analysis method;

FIG. 5B is a schematic diagram to illustrate normalization for the present invention of a psychological stress index measuring system and its analysis method;

FIG. 6A illustrates the distribution map of group-level PPGA for the present invention of a psychological stress index measuring system and its analysis method;

FIG. 6B illustrates the distribution map of group-level HBI for the present invention of a psychological stress index measuring system and its analysis method;

FIG. 7A illustrates the distribution map of individual-level PPGA for the present invention of a psychological stress index measuring system and its analysis method;

FIG. 7B illustrates the distribution map of individual-level HBI for the present invention of a psychological stress index measuring system and its analysis method;

FIG. 8A illustrates the distribution map of combined PPGA for the present invention of a psychological stress index measuring system and its analysis method;

FIG. 8B illustrates the distribution map of combined HBI for the present invention of a psychological stress index measuring system and its analysis method;

FIG. 9A illustrates the cumulative percentage curve of PPGA for the present invention of a psychological stress index measuring system and its analysis method;

FIG. 9B illustrates the cumulative percentage curve of HBI for the present invention of a psychological stress index measuring system and its analysis method;

FIG. 10A illustrates normalized PPGA for the present invention of a psychological stress index measuring system and its analysis method;

FIG. 10B illustrates normalized HBI for the present invention of a psychological stress index measuring system and its analysis method; and

FIG. 11 illustrates the curve of the quantitative psychological stress index for the present invention of a psychological stress index measuring system and its analysis method.

### DETAILED DESCRIPTIONS OF THE PREFERRED EMBODIMENTS

The subject matters, characteristics, and effects with respect to the said or other techniques are clearly indicated by means of detailed descriptions in preferred embodiments and drawings hereinafter.

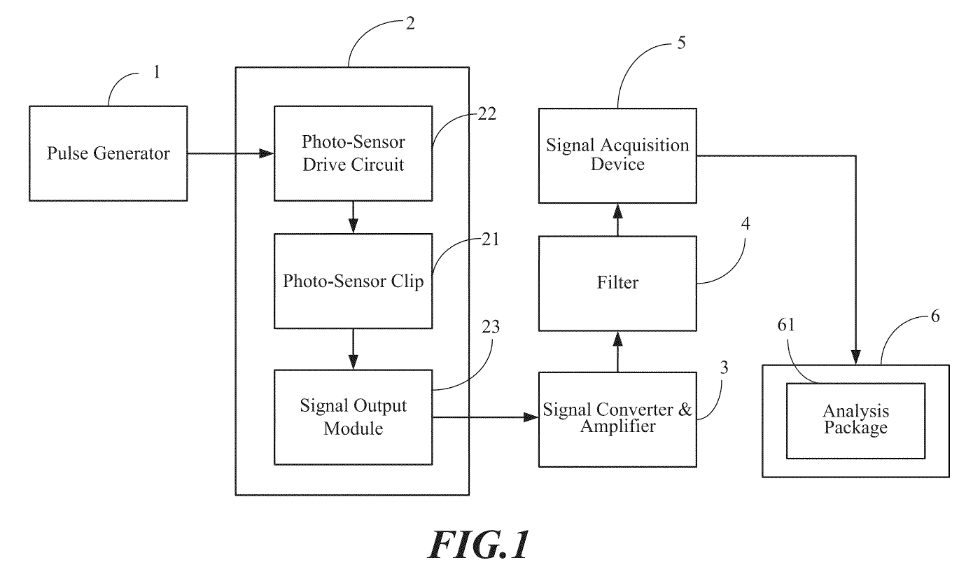

Referring to FIG. 1 which illustrates the overall structure for the present invention of a psychological stress index measuring system and its analysis method wherein the psychological stress index measuring system comprises:

A pulse generator 1 producing a specific-frequency pulse which is driven by alternate red light and infrared light similar to an ON/OFF switch (Electronic device Timer 555 taken as the pulse generator 1 in this embodiment for generation of a 50 Hz pulse).

A transmission-based photo sensor 2 with a photo-sensor clip 21, a photo-sensor drive circuit 22 and a signal output module 23 wherein the photo-sensor drive circuit 22 is electrically connected to both the pulse generator 1 and the photo-sensor clip 21, and the photo-sensor clip 21 is electrically connected to the signal output module 23: As one optical sensor alternately red light and infrared light, the photo-sensor clip 21 is fixed on a user's finger and driven by the photo-sensor drive circuit 22 for red light or infrared light thereof alternately radiated from the user's finger and the user's one analog oxyhemoglobin saturation by pulse oximetry (SpO₂) signal (a current signal in this embodiment) retrieved and exported by the signal output module 23;

A signal converter & amplifier 3 electrically connected to the signal output module 23 of the transmission-based photo sensor 2: The signal converter & amplifier 3 are used in transforming any analog SpO₂ signal exported by the signal output module 23 (i.e., current in this embodiment) to another signal mode (e.g., voltage) for further processing and amplification;

A filter 4 electrically connected to the signal converter & amplifier 3: The filter 4 is effective in filtering noise of any analog SpO₂ signal transformed and amplified by the signal converter & amplifier 3 to obtain either a low-frequency signal or a high-frequency signal;

A signal acquisition device 5 electrically connected to the filter 4 for analog SpO₂ signals from the filter 4 further transformed to digital signals: To avoid any signal distortion and make sure of any pulse signal precisely displayed on the signal acquisition device 5 (a DAQ card in this embodiment; sampling rate=500 Hz which is 10 times faster than the frequency (50 Hz) produced by the pulse generator 1), the sampling rate of the signal acquisition device 5 is twice faster than the rate of any measured analog SpO₂ signal exported by the filter 4;

A processing platform 6 with an analysis package 61: The processing platform 6 could receive any SpO₂ digital signal exported by the signal acquisition device 5 and analyze and quantify any psychological stress level by means of the analysis package 61.

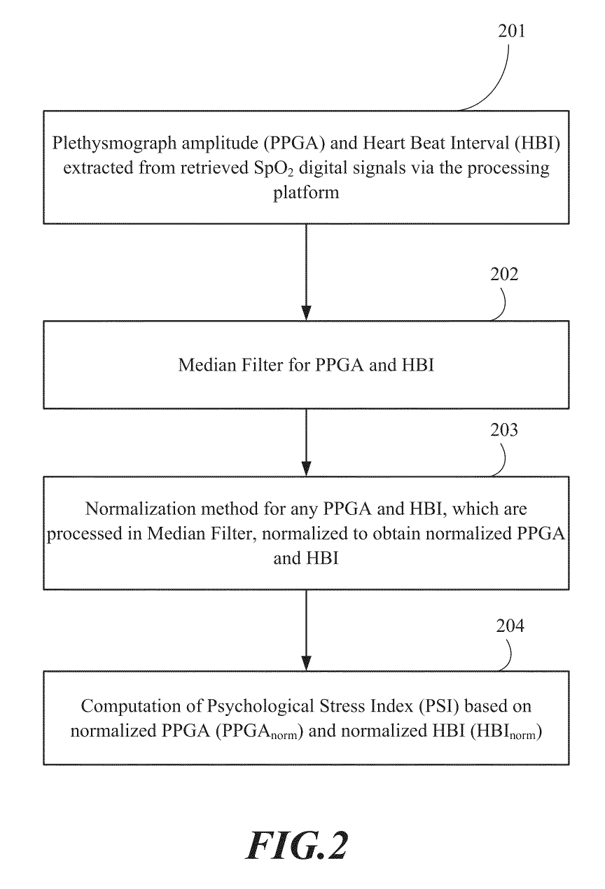

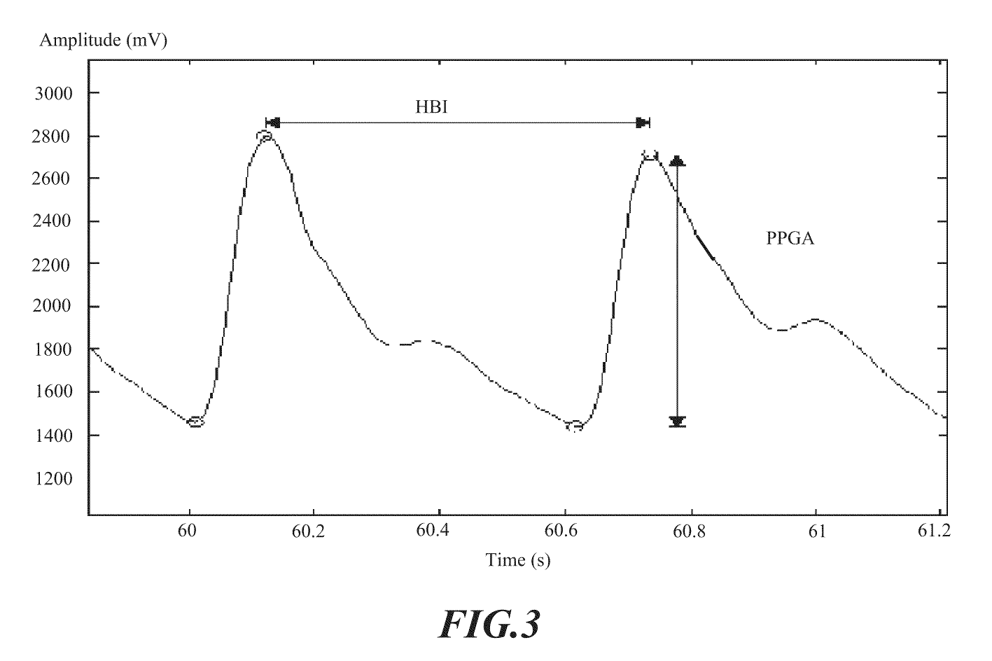

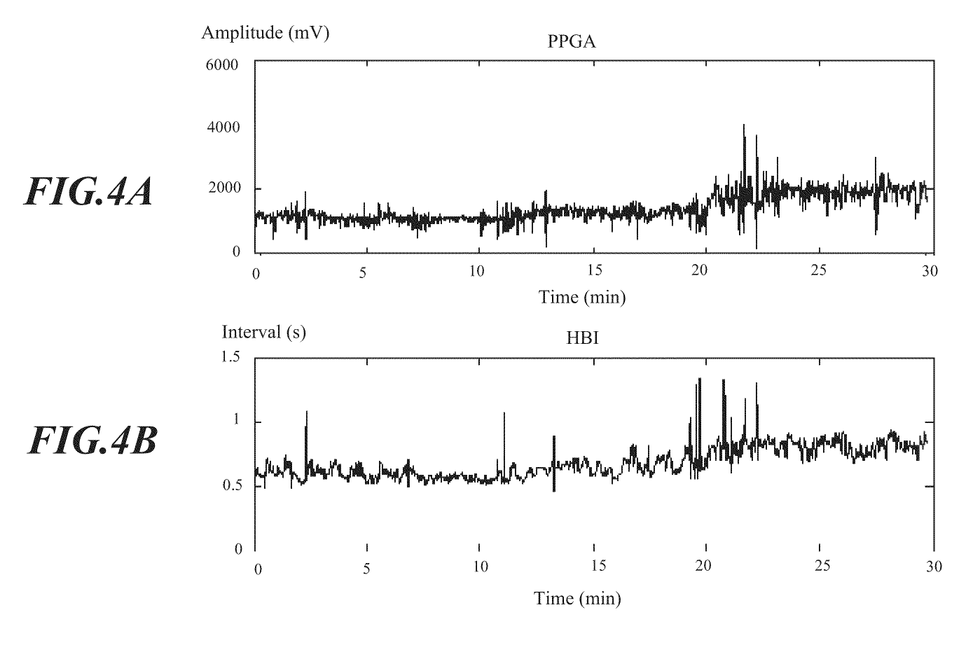

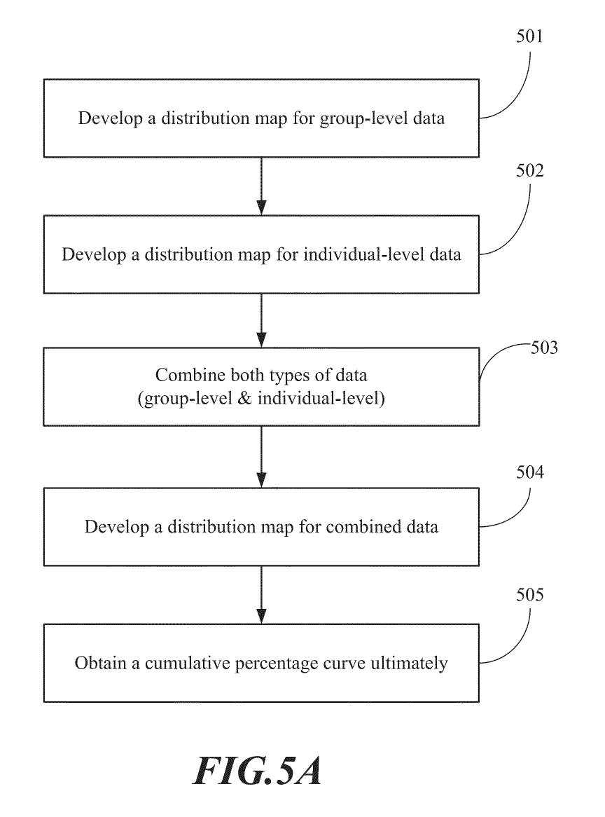

Referring to FIG. 2 which illustrates the analytic flowchart for the present invention of a psychological stress index measuring system and its analysis method with its contents shown as follows:
1. Plethysmograph amplitude (PPGA) and Heart Beat Interval (HBI) extracted from retrieved SpO₂ digital signals via the processing platform: As shown in FIG. 3, FIG. 4A and FIG. 4B, FIG. 3 illustrates definitions of both PPGA and HBI which are further displayed in FIG. 4A (for extracted PPGA) and FIG. 4B (for extracted HBI) respectively with SpO₂ digital signals processed in both different vectors 201;
2. Median Filter for PPGA and HBI: Messages within a selected extent arranged from small to big for further processing by taking a median in place of the central value in the extent thereof and high-frequency noise effectively filtered 202;
3. Normalization method for any PPGA and HBI, which are processed in Median Filter, normalized to obtain normalized PPGA and HBI 203 comprising steps as follows:
   (1) To develop a distribution map for group-level data 501;
   (2) To develop a distribution map for individual-level data 502;
   (3) To combine both types of data hereinabove 503;
   (4) To develop a distribution map for combined data 504; and
   (5) To obtain a cumulative percentage curve ultimately 505.
4. Computation of Psychological Stress Index (PSI) based on normalized PPGA (PPGAₙₒᵣₘ) and normalized HBI (HBIₙₒᵣₘ) with the equation of 100−100×(0.7×PPGAₙₒᵣₘ+0.3×HBIₙₒᵣₘ) 204.

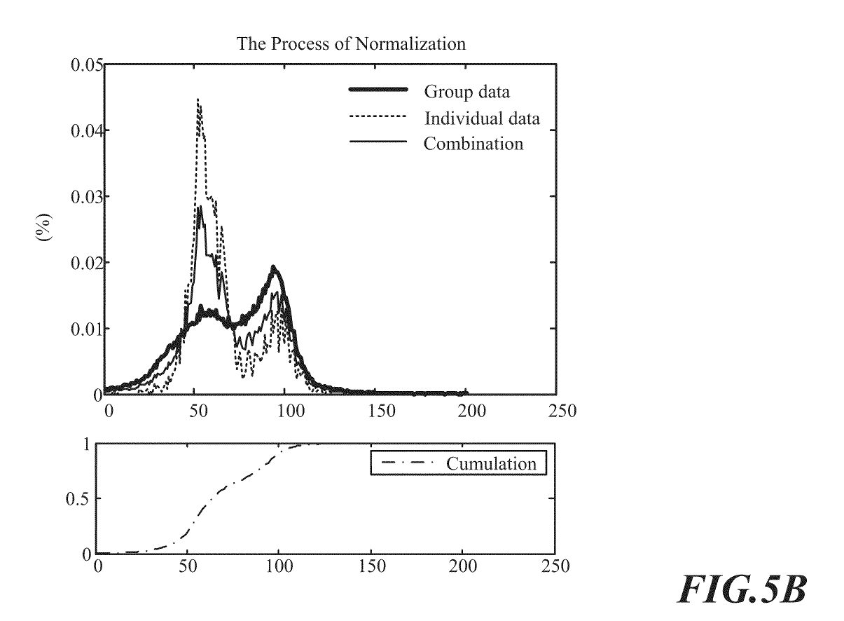

It deserves to be mentioned that one subject-based database including data of all subjects, whether they sustain stress or not, defined as the group-level data and the data related to the subjects sustaining stress as the individual-level data should be developed prior to normalization. During measurement of SpO₂ signals for both the group level and the individual level, theses SpO₂ signals will be designated into 200 regions for plots of distribution maps based on these two types of data (group level & individual level). Then, a cumulative percentage curve based on a combination of these data is able to deliver normalization (FIGS. 5A and 5B).

For the present invention further explained in this embodiment, there are 10 subjects whose mother tongue is not English are arranged to attend meetings for discussing progress of research with their academic advisors and British scholars in English via Skype during three different stages: (1) Stage 1, Preparation (meeting to discuss progress of research not initiated); (2) Stage 2, Presentation in English (divided into the personal presentation and academic advisors' and British scholars' question time); (3) Stage 3, Release (meeting completed).

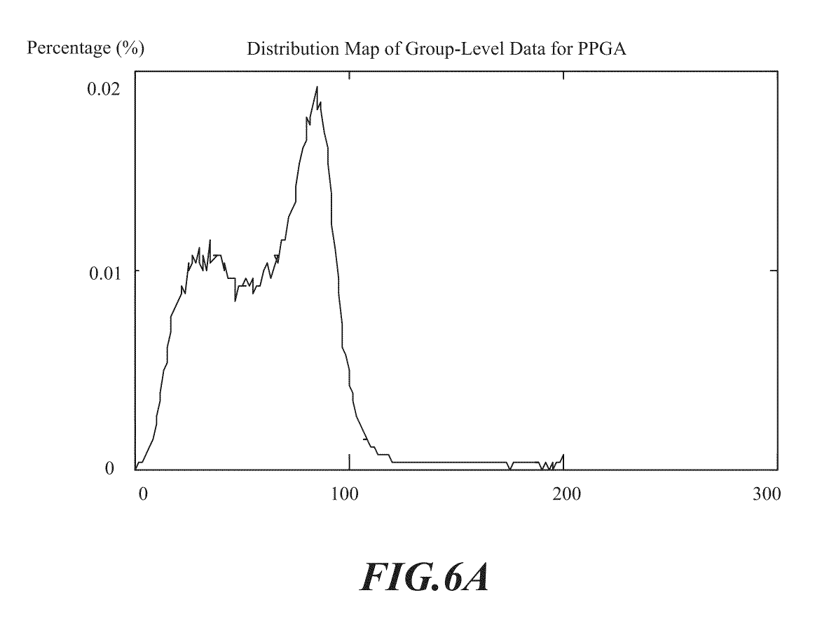

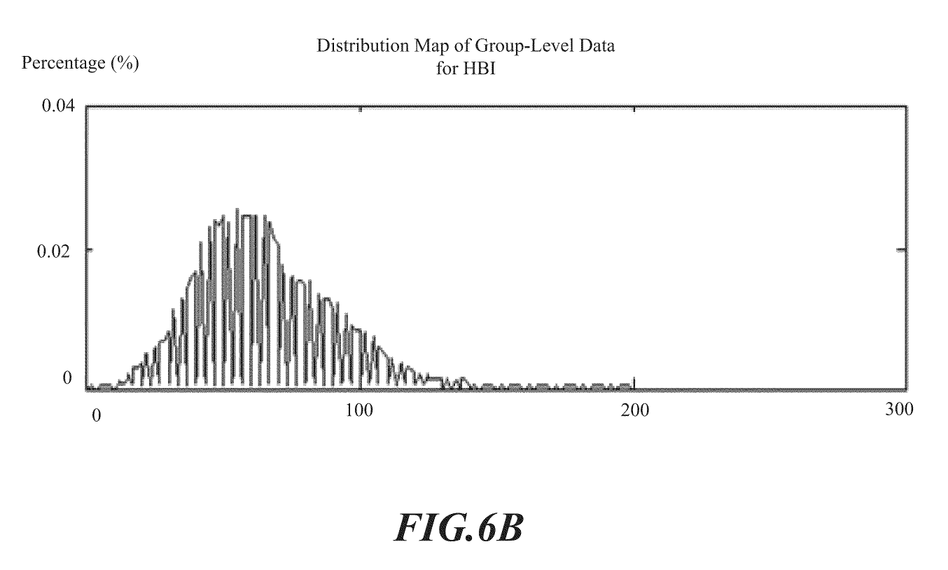

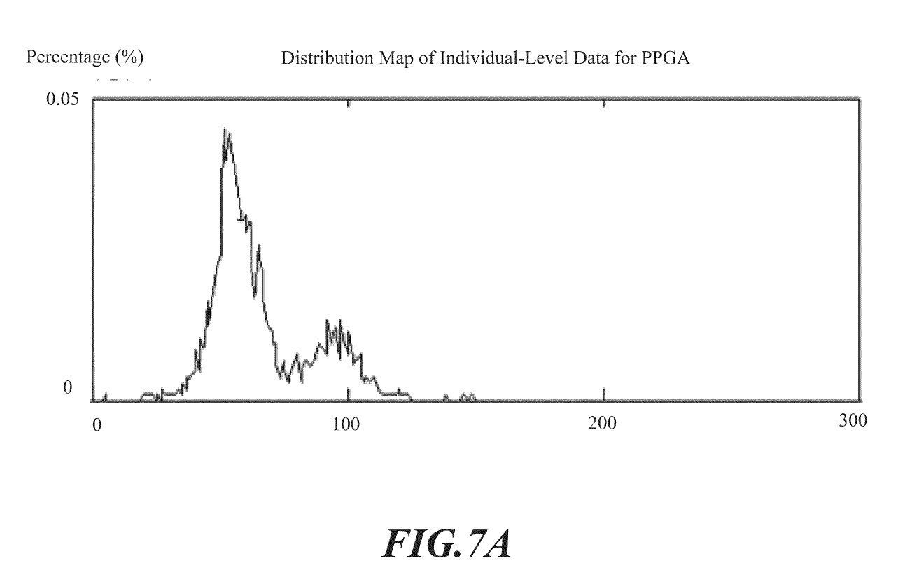

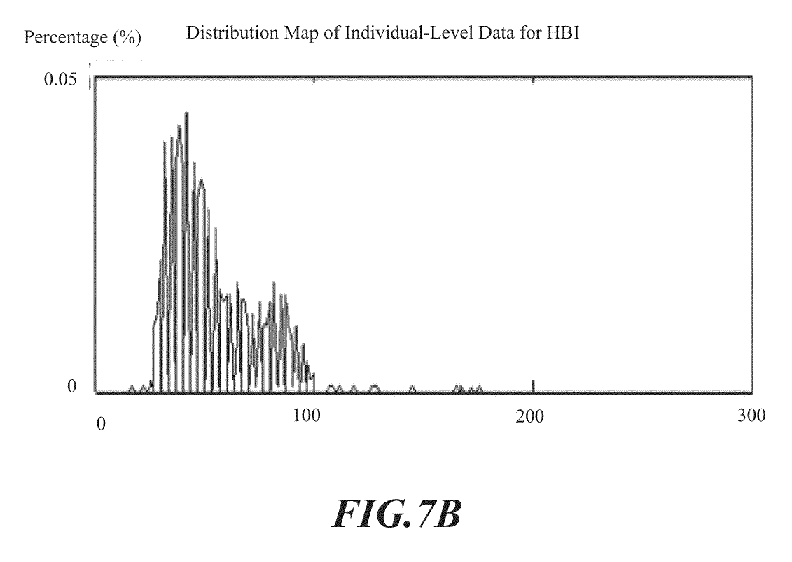

With these 10 subjects' SpO₂ signals measured and received, PPGA (FIG. 4A) and HBI (FIG. 4B) are extracted and used in classifications of group-level data and individual-level data for distribution maps of group-level PPGA (FIG. 6A), group-level HBI (FIG. 6B), individual-level PPGA (FIG. 7A) and individual-level HBI (FIG. 7B).

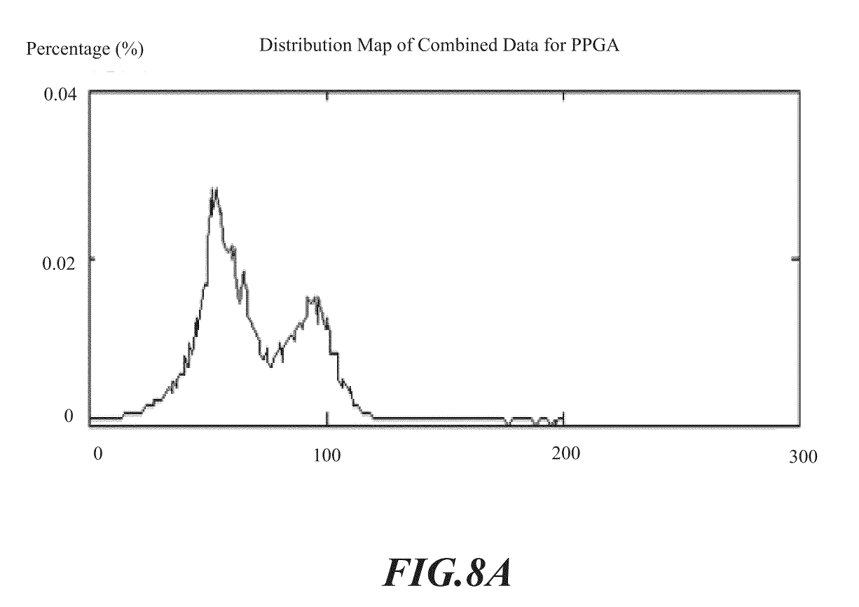

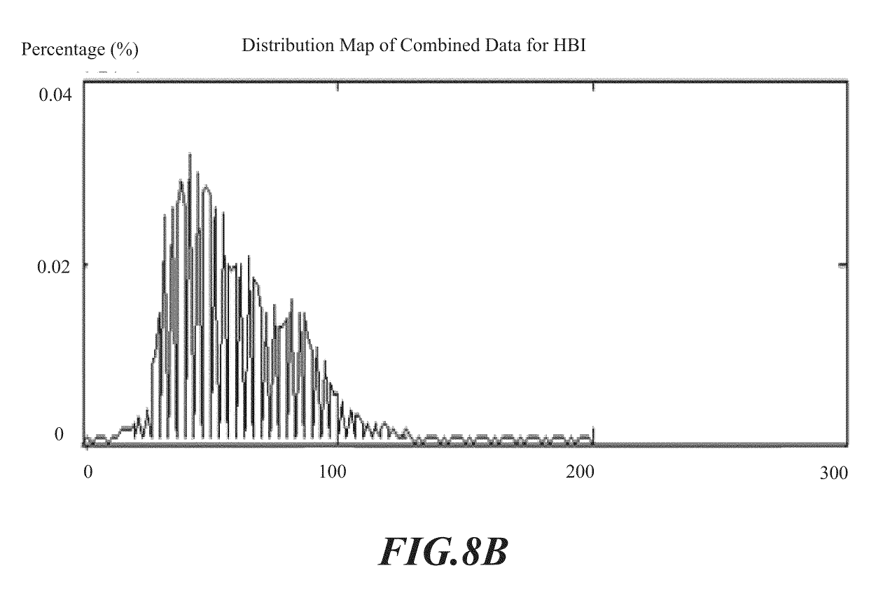

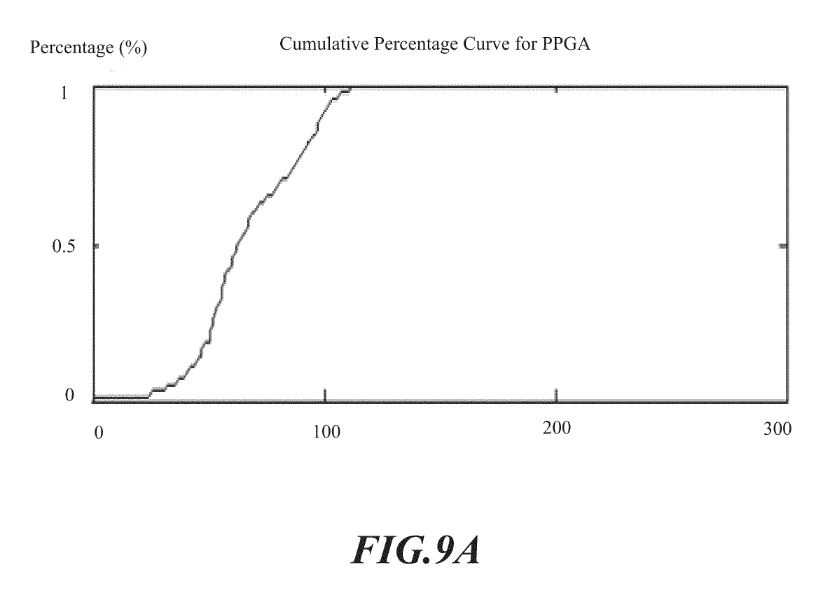

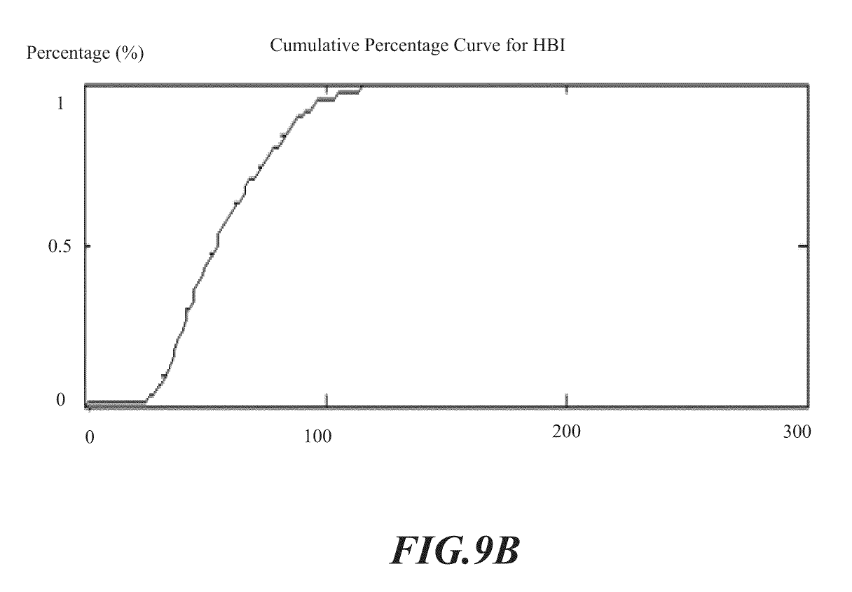

Again, the group-level data with the individual-level data integrated will constitute distribution maps of combined PPGA (FIG. 8A) and combined HBI (FIG. 8B) which are displayed in percentages to develop the cumulative percentage curve of PPGA (FIG. 9A) and the cumulative percentage curve of HBI (FIG. 9B).

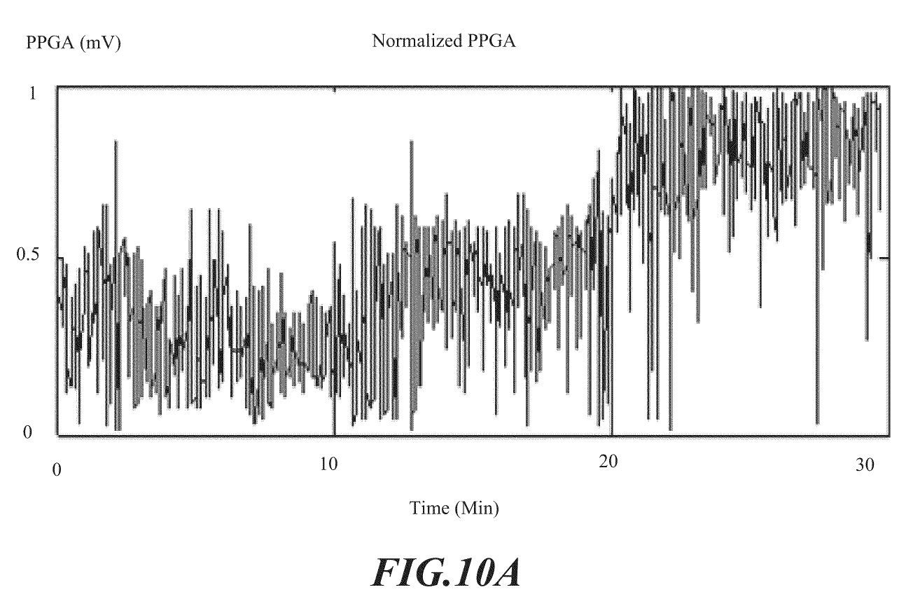

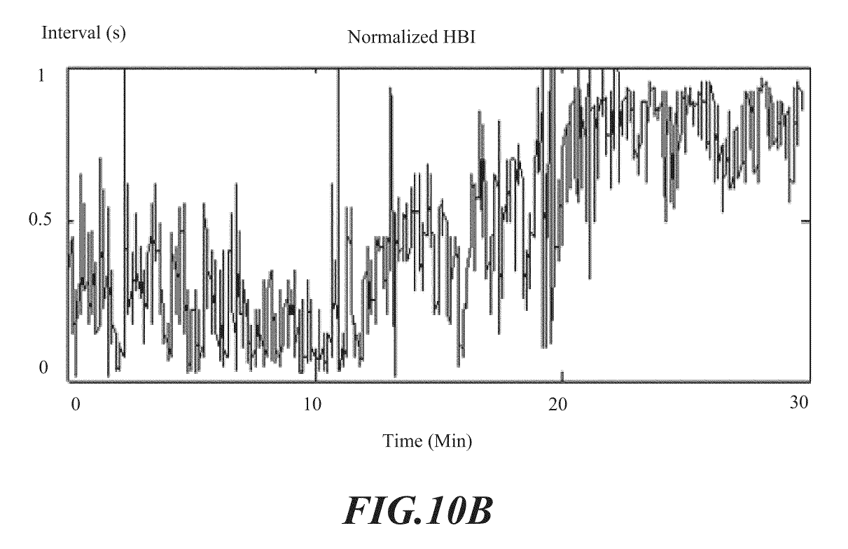

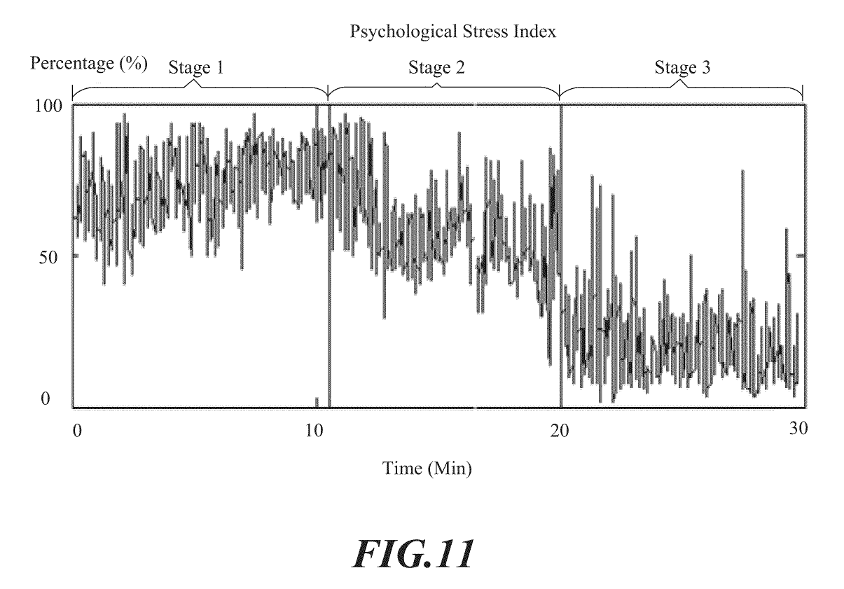

The final results are the normalized PPGA (FIG. 10A) and the normalized HBI (FIG. 10B) by which the data for Psychological Stress Index (FIG. 11) is obtained and three different stages (Stage 1, Stage 2 and Stage 3) are separately differentiated in FIG. 11 to indicate analyzed data in details;

Please refer to Table 1 which illustrates the overall data hereinafter. It can be seen from FIG. 4A to FIG. 10 and Table 1 that the maximum and the minimum Psychological Stress Indices are observed in Stage 2 and Stage 3 respectively. Accordingly, subjects in presentation (Stage 2) are under stress wherein any subject making personal presentations in English expresses higher PSI than in academic advisors' and British scholars' question time. It is thus evident that any subject sustaining more stress during the personal presentation in English compared with academic advisors' and British scholars' question time might be attributed to the subject answering their questions on occasion due to discussions between academic advisors and British scholars taking much time.

Additionally, the data for Psychological Stress Index (PSI) expressed by any subject in Stage 3 (release) sustaining less stress is much lower than that of the subject in Stages 1 & 2 from Table 1 and FIG. 11 because of a significant drop of the curve for PSI from Stage 2 to Stage 3 with the meeting for progress completed (Stage 3).

TABLE 1
Overall data

| | | | | Psychological Stress Index (PSI) | | | | |
|---|---|---|---|---|---|---|---|---|
| | | | | | Stage 2 | | | |
| | | | | | | | Interview | |
| | | Time | | | | | with | |
| | | (Personal | | | | Personal | Academic | |
| Subject | Gender/Age | Presentation) | Heartbeat | Stage 1 | Overall | Presentation | advisors | Stage 3 |
| 1 | Male/24 | 31.4 | 82 | 61.6 | 59.1 | 69.0 | 48.5 | 9.9 |
| 2 | Female/21 | 33.9 | 66 | 53.8 | 46.3 | 48.3 | 44.2 | 36.0 |
| 3 | Male/23 | 14.5 | 75 | 33.3 | 53.0 | 68.3 | 37.7 | 22.7 |
| 4 | Male/23 | 15.2 | 84 | 31.7 | 53.0 | 67.2 | 40.1 | 22.4 |
| 5 | Male/24 | 27.6 | 81 | 53.6 | 66.3 | 75.3 | 57.2 | 25.8 |
| 6 | Female/22 | 22.0 | 78 | 66.0 | 75.4 | 71.8 | 79.0 | 12.7 |
| 7 | Female/21 | 20.0 | 77 | 18.4 | 63.7 | 68.1 | 59.2 | 21.1 |
| 8 | Male/24 | 12.5 | 81 | 64.8 | 56.4 | 59.8 | 53.0 | 30.4 |
| 9 | Male/25 | 13.5 | 87 | 45.1 | 58.8 | 69.0 | 48.5 | 12.6 |
| 10 | Female/21 | 18.5 | 83 | 60.4 | 61.8 | 70.3 | 53.2 | 18.9 |

| | | | | | | |
|---|---|---|---|---|---|---|
| Mean ± Standard Deviation | 79 ± 5.9 | 48.9 ± 16.2 | 59.4 ± 8.1 | 66.7 ± 7.6 | 52.0 ± 11.7 | 21.3 ± 8.2 |

In contrast to other prior arts, a psychological stress index measuring system and its analysis method provided in the present invention has the following advantages:
1. The present invention featuring SpO₂ signals noninvasively measured for development of one index to evaluate psychological stress is effective in obtaining SpO₂ information and quantifying the psychological stress level and contributes to a specific psychological evaluation mode.
2. The present invention measuring SpO₂ signals with a noninvasive system is characteristic of convenience, portability and operability and is applicable to an elder or a patient with a long-term demand to observe SpO₂ signals.

The purpose of disclosing the preferred embodiment is to clearly describe the characteristics and spirit included in the present invention but not to restrict it. Conversely, the purpose is to further deliver a variety of changes and any equivalent arrangement covered in claims of the present invention.

What is claimed is:

1. A psychological stress index measuring system comprising:
A pulse generator producing a pulse with a specific frequency which is driven by alternate radiation similar to a switch;
A transmission-based photo sensor with a photo-sensor jig, a photo-sensor drive circuit, and a signal output module wherein the photo-sensor drive circuit is electrically connected to both the pulse generator and the photo-sensor jig and the photo-sensor jig is connected to the signal output module for a user's SpO₂ (oxyhemoglobin saturation by pulse oximetry) signals received and exported by the signal output module;
A signal converter & amplifier electrically connected to the transmission-based photo sensor's signal output module and transforming an analog SpO₂ signal exported by the signal output module to one signal mode which could be processed and further amplified;
A filter electrically connected to the signal converter & amplifier and filtering noise of any analog SpO₂ signal transformed and amplified by the signal converter & amplifier;
A signal acquisition device electrically connected to the filter for any analog SpO₂ signal, which is exported by the filter, transformed to a digital signal;
A processing platform with one analysis package installed which is used to quantify the psychological stress level based on any received digital SpO₂ signal transmitted from the signal acquisition device.

2. The psychological stress index measuring system according to claim 1 wherein the photo-sensor jig is an optical sensor alternately radiating red light & infrared and fixed on a user's finger.

3. The psychological stress index measuring system according to claim 1 wherein the pulse generator could produce a pulse exported into the photo-sensor drive circuit which drives the photo-sensor jig for red light & infrared alternately radiated from the user and any analog SpO₂ signal from the user received by the signal output module.

4. A psychological stress index analysis method featuring:
(1) PPGA and HBI extracted from any retrieved digital SpO₂ signal by the processing platform;
(2) Median filter for PPGA and HBI;
(3) Normalization of any PPGA and HBI which have been processed in Median Filter for any normalized PPGA and HBI; and
(4) Computation of Psychological Stress Index (PSI) based on the normalized PPGA and HBI.

5. The psychological stress index analysis method according to claim 4 wherein the digital SpO₂ signals are divided into two vectors for extraction of PPGA and HBI.

6. The psychological stress index analysis method according to claim 4 wherein the median filter is characteristic of high-frequency noise effectively filtered by arranging messages within a selected extent from small to big and taking a median in place of the central value within the extent thereof.

7. The psychological stress index analysis method according to claim 4 wherein a subject-based database including data of all subjects, whether they sustain stress or not, defined as the group-level data and the data related to the subjects sustaining stress as the individual-level data need to be developed prior to normalization.

8. The psychological stress index analysis method according to claim 7 wherein the normalization comprises the following steps:
(1) To develop a distribution map for group-level data;
(2) To develop a distribution map for individual-level data;
(3) To combine both types of data hereinabove;
(4) To develop a distribution map for combined data; and
(5) To obtain a cumulative percentage curve ultimately.

9. The psychological stress index analysis method according to claim 4 wherein the equation for the Psychological Stress Index is 100−100×(0.7×normalized PPGA+0.3×normalized HBI).
